(19) United States
(12) Patent Application Publication
(10) Pub. No.: US 2010/0249798 A1
(43) Pub. Date: Sep. 30, 2010

(54) INSTRUMENT FOR PLACING A BONE SCREW, NOTABLY A SO CALLED "POLYAXIAL" SCREW OF VERTEBRAL OSTEOSYNTHESIS EQUIPMENT

(76) Inventors: Denys Sournac, Reyrieux (FR); David Ryan, Collonges Au Mont D'Or (FR)

Correspondence Address: DEFILLO & ASSOCIATES, INC., P.O. Box 14104, Clearwater, FL, 33766, US

(21) Appl. No.: 12/741,210

(22) PCT Filed: Nov. 5, 2008
(86) PCT No.: PCT/IB2008/054606

§ 371 Date: May 4, 2010

(30) Foreign Application Priority Data
Nov. 5, 2007 (FR) 0707752

(51) Int. Cl. A61B 17/56

(52) U.S. Cl. 606/104

(57) ABSTRACT

This instrument (1) includes a first component (3) intended to engage with the threaded proximal portion of the screw, a second component (2) intended to engage with the threaded distal portion of the screw, and means (4) for driving the first component (3) into rotation relatively to the second component (2). The driving means (4) include an actuation member (26, 27) for performing the driving into rotation. According to the invention, the actuation member (26, 27) is rotationally bound to the first member (3) via releasable connection means (33, 36), being released beyond a torque threshold exerted on this actuation member (26, 27) in the direction of rotation corresponding to the mounting of the screw on the instrument (1).

### CROSS REFERENCE TO RELATED APPLICATION

This application is a national stage entry of PCT/IB2008/054606 filed Nov. 5, 2008, under the International Convention claiming priority over French Application No. 0707752 filed Nov. 5, 2007.

### FIELD OF THE INVENTION

The present invention relates to an instrument for placing a bone screw, notably a so-called “polyaxial” screw of vertebral osteosynthesis equipment.

### BACKGROUND OF THE INVENTION

A piece of vertebral osteosynthesis equipment frequently comprises fixing screws, each of which includes a distal threaded portion, intended to be inserted in a vertebra generally at a pedicle, and a proximal threaded portion, used for mounting the piece of equipment on the vertebra. This proximal threaded portion is frequently jointed relatively to the distal threaded portion, the screw being then currently designated as “polyaxial” screw.

Such a polyaxial screw is set into place by means of an instrument comprising an internal tube, an external tube and means for driving the internal tube into rotation relatively to the external tube. The internal tube comprises a tapped distal end, intended to be engaged with the proximal threaded portion of the screw, and the external tube comprises a distal end intended to be engaged with a shoulder formed by the distal threaded portion of the screw; the rotational driving means comprise a knurl mounted on the internal tube. In practice, the distal tapped end of the internal tube is brought into engagement with the proximal threaded portion of the screw and then the knurl is actuated in rotation in the direction for screwing the internal tube on the threaded proximal end so as to bring the shoulder formed by the distal threaded portion of the screw into engagement with the distal end of the external tube. The thereby generated axial tension on the screw allows the instrument to perfectly grasp this screw, with blocking of the screw in rotation relatively to the instrument. The latter may then be used for placing the screw in the pedicle of a vertebra, without the proximal threaded portion of the screw being an obstacle to its placement.

With the existing instrument, the significant drawback of rotational blocking of the internal tube-knurl assembly after placement of the screw is reported, frequently requiring that the practitioner use a key for untightening this assembly in order to release the screw after placement. It is difficult for the practitioner to determine a tightening of the knurl both sufficient for ensuring perfect connection of the screw to the instrument and insufficient for achieving blocking of the knurl.

### SUMMARY OF THE INVENTION

The main object of the present invention is to find a remedy to this drawback.

Another object of the invention is to achieve this main object with an instrument structure which remains easily sterilizable without any substantial increase in the manufacturing cost of the instrument.

The relevant instrument comprises in a way known per se, a first component intended to engage with the threaded proximal portion of the screw, a second component intended to engage with the threaded distal portion of the screw, and means for driving the first component into rotation relatively to the second component, so that, in a direction of rotation corresponding to the mounting of the screw on the instrument, said first component may be brought so as to engage with the threaded proximal portion of the screw, and the screw to engage with said second component, these driving means comprising an actuation member for performing said driving into rotation.

According to the invention, said actuation member is rotationally bound to said first member via releasable connection means, being released beyond a torque threshold exerted on this actuation member in the direction of rotation corresponding to the mounting of the screw on the instrument.

With the instrument according to the invention, it is thereby possible to avoid that a tightening torque be exerted on the actuation member which may lead after placement of the screw to an impossibility of manually untightening this actuation member.

Preferably, the releasable connection means comprise:

a first series of teeth laid out on said first component or on a part integral with this first component;

a second series of teeth laid out on the actuation member, and

holding means capable of maintaining the teeth of both of these series of teeth, in engagement with each other until said tightening torque is reached and allowing these teeth to be disengaged when the tightening torque is reached.

Said maintaining means may notably comprise an elastic member normally maintaining the teeth engaged with each other and the elastic deformation of which allows the teeth to become disengaged.

Preferably,

said first series of teeth is laid out in the proximal end of said first component or on a part integral with this first component;

said actuation member has the shape of a bushing which will cap the proximal end of the first component or of said part integral with this first component, comprising a bottom in which said second series of teeth is laid out, and

said proximal end or said part and said bushing form respective abutment surfaces between which a spring is interposed, forming said maintaining means.

According to a preferred embodiment of the invention, in this case:

said part forms a shoulder forming a first abutment surface;

the actuation member comprises an external part including said bottom and comprising a tapped peripheral wall and an inner part comprising a threaded peripheral wall and an internal shoulder forming a second abutment surface.

The actuation member is thus made up by engaging said inner part onto the proximal end of the first component, by placing the spring between the respective abutment surfaces, and then by screwing said external part onto said inner part.

Said inner part may form a distal housing receiving an O-ring gasket opposing the inflow of fluids into the interior of the actuation member.

The housing receiving the spring is thus protected with regard to penetration of such fluids.

Said first component advantageously comprises a distal tube, the distal end of which is tapped in order to engage with the proximal threaded portion of the screw, this tube being connected on the proximal side, to a proximal part, coaxial therewith, comprising said first series of teeth laid out in its proximal end.

Said second component advantageously comprises a distal tubular portion in which said first component is engaged and a cage-shaped proximal portion delimiting a housing in which said actuation member is found.

### BRIEF DESCRIPTION OF THE DRAWINGS

The invention will be well understood, and other features and advantages thereof will become apparent with reference to the appended schematic drawing, illustrating as a non-limiting example, a preferred embodiment of the instrument to which it relates.

FIG. 1 is a perspective view thereof;

FIG. 2 is a longitudinal sectional view thereof;

FIG. 3 is view thereof, similar to FIG. 2, at an enlarged scale;

FIG. 4 is an exploded perspective view thereof;

FIG. 5 is a perspective view of a proximal part of a first component which it comprises, and

FIG. 6 is a perspective view of an actuation member which it comprises.

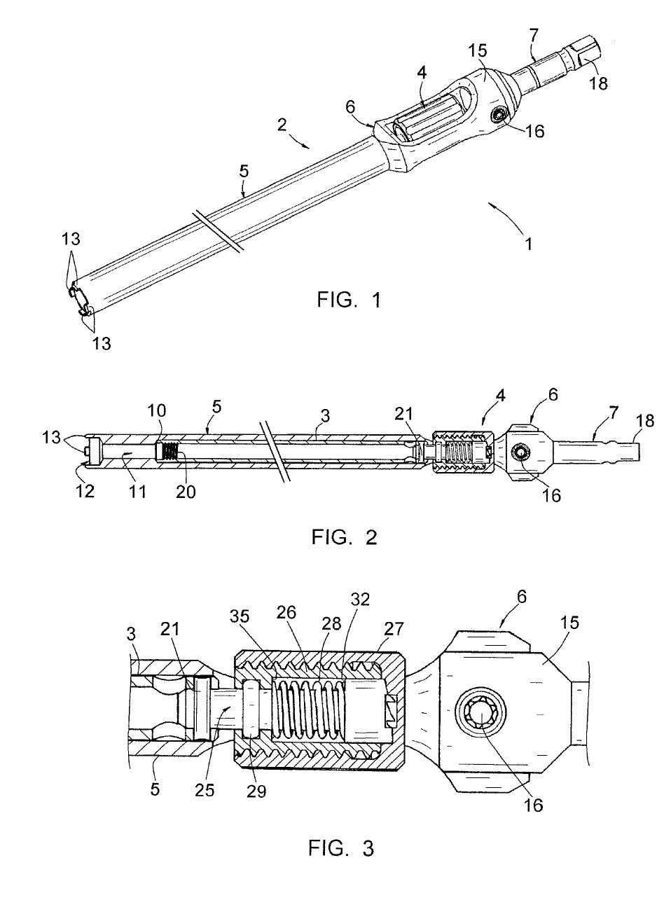

### DETAILED DESCRIPTION OF THE INVENTION

The figures illustrate an instrument 1 for placing a so-called “polyaxial” bone screw of vertebral osteosynthesis equipment.

This screw is as described in French Patent Application No. 07 07468. It includes a distal threaded portion and a proximal threaded pin jointed relatively to the distal threaded portion.

The latter comprises a threaded body and a proximal head. The threaded body is intended to be inserted into a vertebra, generally at a pedicle. The head forms a proximal cavity intended to receive with jointing, the proximal threaded pin and comprises a proximal collar in which four radial notches are laid out, positioned at 90 degrees from each other.

The proximal threaded pin comprises a threaded rod with which it may be assembled with other parts which the piece of equipment comprises (in particular to a stirrup for connecting to a longitudinal linking rod) and a distal bulging portion intended to be received with jointing, in said proximal cavity of the distal threaded portion.

This distal bulging portion and this proximal cavity at their periphery have locking shapes so that the pins may be locked in rotation relatively to the distal threaded portion while allowing the pin to be jointed relatively to this distal threaded portion. These locking shapes may notably be hexagonal with rounded edges as described in French Patent Application No. 07 07468.

As this is illustrated in the figures of the present patent application, the instrument 1 comprises an external body 2, an internal tube 3 and an assembly 4 for maneuvering the internal tube 3 relatively to the external body 2.

The external body 2 comprises a distal tubular portion 5, an intermediate portion 6 and a proximal portion 7.

The distal tubular portion 5 contains the internal tube 3, which is engaged into it in an adjusted way with possibility of rotation. It forms a distal shoulder 10, against which the distal end of the internal tube 3 is intended to abut. It also forms a distal bore 11 and, at its distal end, a cavity 12 and four teeth 13 positioned at 90 degrees from each other. The distal bore 11 is intended to receive the proximal threaded pin of the screw, which may engage by screwing with a tapped thread 20 positioned in the distal end of the internal tube 3; the cavity 12 as for it receives the head of the distal threaded portion of the screw until the distal end of the distal tubular portion 5 bears against the collar of the screw. During this bearing action, the teeth 13 will engage into the notches which this collar comprises, achieving engagement of the screw and of the instrument 1.

The intermediate portion 6 forms a body with the distal tubular portion 5 and is cage-shaped, delimiting a housing for receiving the maneuvering assembly 4. On the proximal side, the intermediate portion 6 comprises an end 15 pierced with an axial bore, for the engagement of the internal tube 3 and of the maneuvering assembly 4 through it, and with a transverse bore receiving a screw spindle 16 for mounting the proximal portion 7.

The proximal portion 7 comprises a proximal square-shaped end 18, allowing the instrument 1 to be maneuvered in rotation for screwing or unscrewing the screw, and a distal portion pierced with a transverse bore for receiving the spindle 16.

The internal tube 3 comprises, in addition to the tapped thread 20, a transverse bore laid out in its proximal end, intended to receive a transverse spindle 21 for rotatably connecting this tube 3 to the maneuvering assembly 4.

The maneuvering assembly 4 has an internal rod 25, a threaded internal part 26, a tapped external part 27, a spring 28 and a seal gasket 29.

The internal rod 25 comprises a cylindrical body 30 and a head 31. The distal end of the body 30 comprises a through-hole intended to be crossed by the spindle 21 in order to allow the rotary connection of the part 25 and of the internal tube 3. The head 31 has a widened diameter relatively to the diameter of the body 30 so that it delimits with the latter a shoulder 32 turned towards the distal side.

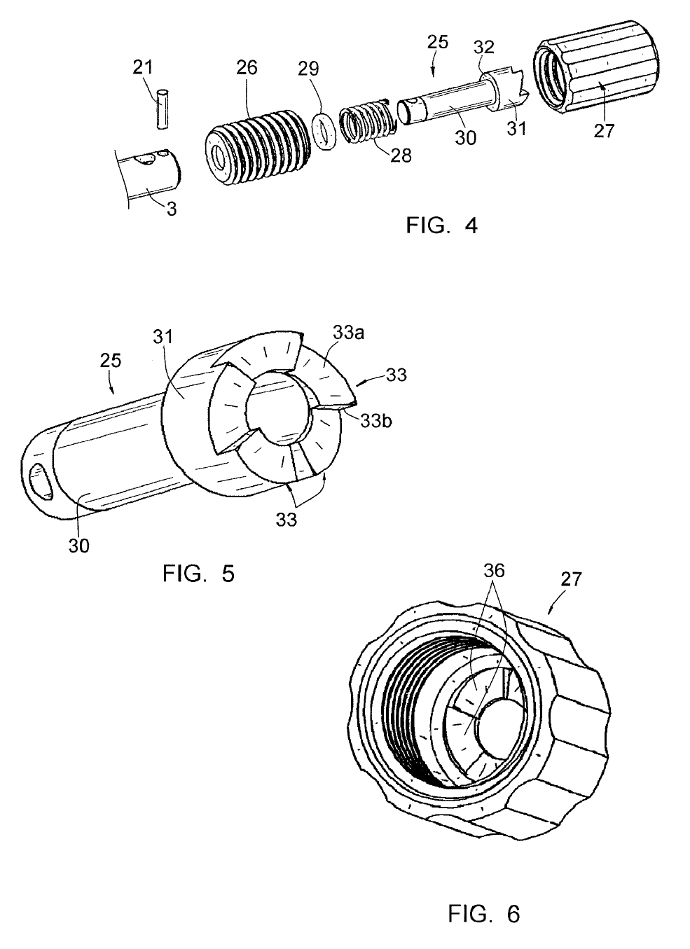

At its proximal end, this head 31 comprises, as this is more particularly shown in FIG. 4, a series of five teeth 33 laid out on the whole of its periphery, each tooth 33 comprising a tilted flank 33a forming the proximal end of the head 31, and a straight flank 33b, positioned parallel to the longitudinal axis of the part 25. The tilt of each tilted flank 33a relative to this longitudinal axis is such that it forms an angle of the order of 70-80 degrees with this axis.

The threaded internal part 26 comprises a distal axial bore and a proximal axial bore, the distal bore being intended to receive in an adjusted way, the body 30 of the internal rod 25, while the proximal bore is intended to receive in an adjusted way the head 31 of this same rod 25. The part 26 thereby forms a shoulder 35 turned towards the proximal side. At its wall delimiting the distal bore, the part 26 comprises a groove for receiving the seal gasket 29.

The tapped external part 27 has the shape of a bushing, i.e. it comprises a tapped peripheral wall, allowing it to be screwed onto the threaded internal part 26, and a bottom. This bottom comprises, as this is most particularly visible in FIG. 5, a series of teeth 36 of the same shape as the teeth 33, able to cooperate with the latter in order to form releasable connecting means, the operation of which will be explained later on.

The outer face of the tapped external part 27 is ribbed, so as to promote its gripping by hand for exerting a tightening or untightening torque on this part.

The spring 28 is interposed between the shoulders 32 and 35 and allows the head 31 to be pressed against the bottom of the tapped external part 27 so that the teeth 33 are normally maintained engaged with the teeth 36 and so there exists a rotary connection between the part 27 and the internal tube 3.

In practice, the pin of the screw is inserted into the bore 11 and the tube 3 is screwed onto it by rotationally actuating the part 27, until the head which this screw comprises, is introduced into the cavity 12 and the collar which this screw also comprises, abuts against the distal end of the distal tubular portion 6. When this abutment is achieved, possible continuation of the manoeuvre in rotation of the part 27 in the tightening direction will have the effect of sliding the tilted flanks of the teeth 36 against the corresponding tilted flanks 33a of the teeth 33, causing an axial displacement of the part 26-part 27 assembly relatively to the rod 25 against the elastic force of the spring 28, until these teeth 36, 33 escape. The rotary connection of the part 27 relatively to the tube 3 is thus released beyond a torque threshold exerted on the part 27 in the direction of rotation corresponding to the mounting of the screw on the instrument 1, by this release it is possible to prevent a tightening torque from being exerted on the part 27, which may lead after placement of the screw, to an impossibility of manually unscrewing this part 27.

When the part 27 is manoeuvred in the unscrewing direction, the straight flanks of the teeth 26 bear against the straight flanks 33b of the teeth 33 and ensures the unscrewing of the internal tube 3 relatively to the proximal pin of the screw.

As this appears in the foregoing, the invention provides an instrument for placing a bone screw, notably a so-called “polyaxial” screw of vertebral osteosynthesis equipment, having determining advantages as compared with homologous instruments of the prior art.

The invention was described above with reference to an embodiment described as a pure example. It is obvious that it is not limited to this embodiment but that it extends to all embodiments covered by the appended claims herein.

1- An instrument (1) for placing a bone screw including a distal threaded portion, notably a so-called “polyaxial” screw of vertebral osteosynthesis equipment, comprising:
a first component (3) intended to engage with the threaded proximal portion of the screw,
a second component (2) intended to engage with the threaded distal portion of the screw, and
means (4) for driving the first component (3) into rotation relatively to the second component (2), so that, in a direction of rotation corresponding to the mounting of the screw on the instrument (1), said first component (3) may be brought so as to engage with the threaded proximal portion of the screw, and the screw to engage with said second component (2), these driving means (4) comprising an actuation member (26, 27) for performing said driving into rotation;
wherein said actuation member (26, 27) is rotationally bound to said first member (3) via releasable connection means (33, 36), being released beyond a torque threshold exerted on this actuation member (26, 27) in the direction of rotation corresponding to the mounting of the screw on the instrument (1).

2- The instrument (1) according to claim 1, wherein the releasable connection means comprise:
a first series of teeth (33) laid out on said first component (3) or on a part (25) integral with this first component (3);
a second series of teeth (36) laid out on the actuation member (26, 27), and
holding means (28) capable of maintaining the teeth (33, 36) of both of these series of teeth, in engagement with each other until said tightening torque is reached and allowing these teeth to be disengaged when the tightening torque is reached.

3- The instrument (1) according to claim 2, wherein said maintaining means comprise an elastic member (28) normally maintaining the teeth (33, 36) engaged with each other and the elastic deformation of which allows the teeth to become disengaged.

4- The instrument (1) according to claim 2 wherein
said first series of teeth (33) is laid out in the proximal end of said first component (3) or on a part (25) integral with this first component;
said actuation member (26, 27) has the shape of a bushing which will cap the proximal end of the first component (3) or of said part (25) integral with this first component, comprising a bottom in which said second series of teeth (36) is laid out, and
said proximal end or said part (25) and said bushing form respective abutment surfaces (32, 35) between which a spring (28) is interposed, forming said maintaining means.

5- The instrument (1) according to claim 4, wherein
said part (25) forms a shoulder (22) forming a first abutment surface; and
the actuation member (26, 27) comprises an external part (27) including said bottom and comprising a tapped peripheral wall and an inner part (26) comprising a threaded peripheral wall and an internal shoulder (35) forming a second abutment surface.

6- The instrument (1) according to claim 5, wherein said inner part (26) forms a distal housing receiving an O-ring gasket (29) opposing the inflow of fluids into the interior of the actuation member (26, 27).

7- The instrument (1) according to claim 1, wherein said first component (3) comprises a distal tube, the distal end of which is tapped in order to engage with the proximal threaded portion of the screw, this tube being connected on the proximal side, to a proximal part (25), coaxial therewith, comprising said first series of teeth (33) laid out in its proximal end.

8- The instrument (1) according to claim 1, wherein said second component (2) comprises a distal tubular portion (5) in which said first component (3) is engaged and a cage-shaped proximal portion (6) delimiting a housing in which said actuation member (26, 27) is found.
